(19) United States
(12) Patent Application Publication
(10) Pub. No.: US 2008/0292665 A1
(43) Pub. Date: Nov. 27, 2008

(54) Simple mechanical procedure and product for deterring substance abuse

(76) Inventor: John C. Kulli, Hamlin, NY (US)

Correspondence Address: PETER I. LIPPMAN, 17900 MOCKINGBIRD DRIVE, RENO, NV, 89508, US

(21) Appl. No.: 11/807,355

(22) Filed: May 25, 2007

(51) Int. Cl. A61K 9/00

(52) U.S. Cl. 424/400

(57) ABSTRACT

Simple mechanical methods improve safety of a substance that is at risk for abuse. The substance is made in a specified form that tends to deter conversion to powder: and in this form commercially distributed, e.g. wholesale or retail. Preferably that form is enclosed in a harmless container. Preferably the method also includes putting the substance into or onto a harmless carrier. Preferably the carrier does not dissolve in. It may e oil or solid—for example paper or similarly thin medium, or a sponge or other medium that has generally course cellular structure.

### BACKGROUND

Many common substances that are widely used in the U.S. and elsewhere, for legitimate purposes, are unfortunately also frequently diverted for illicit use. One frequent mode of abuse is snorting. A substance that is solid can be ground mechanically into a fine dust and then snorted, thereby introducing the powder into the nose, where it is rapidly absorbed.

This method of use bypasses the stomach and the liver, resulting in a far more rapid and potent effect of the same amount of substance when ingested by means other than snorting. Snorting substances intended to be taken by mouth is dangerous for several reasons.

First, such substances often are sold mixed with other ingredients—designed to improve the easy use of the substance. These can include, according to the manufacturer, various harmless ingredients. Although these ingredients are harmless when taken by mouth, they can have serious health effects when snorted.

Second, delivery of such substances into the system is far more effective. This poses a real risk of harmful effects upon a person's body.

As noted above, some solid substances are easily prepared for snorting by simply grinding them into powder. Among college students this is frequently done by placing a piece of the substance 11 (FIG. 1) in the depression in the bottom of a small bottle 12, and crushing the piece into fragments 11′ with a coin 13.

Often or usually, crushing continues until much of the piece has been reduced to a finer, snortable powder 11″ (FIG. 3). This material is brought near nostrils 15 of the substance abuser, who then inhales to draw a stream 14 of the powder into the nostrils.

Dangerous substances can also be abused by injection. This is accomplished after dissolving the powdered piece in water, aided by heating the water. The resulting mixture of the mechanically produced substance is then injected into the body.

A liquid form of some of the subject substances exists commercially, but it is not formulated to discourage mechanical alteration. The liquid form is relatively much harder than the powder to administer, for legitimate purposes, and therefore its legitimate use is disfavored in some environments. This is particularly true for some such substances (e.g. some substances that are medications), in elementary school—where accurate injections are difficult or impossible. The liquid form also is susceptible to diversion by alteration.

Recently two new products that include such substances have come onto the market. One is a so-called “chewable” product.

Notwithstanding the use of the term “chewable”, the new preparation does no more than merely bulk up the product. A substance abuser can still mechanically pulverize the substance, enabling it to be snorted effectively. It appears that no thought has been given to the common knowledge, among young people, of how easy it is to mechanically alter such substances to allow them to be snorted.

The other substance newly available is in a label-like form which, itself, cannot be pulverized for snorting purposes. For at least two reasons, however, it does not solve the abuse problems described above.

The label-like form can also present cause, in the case of children bearing the product, for taunting and harassment by others. This makes it possible or probable that a child will remove the device to avoid unwanted attention.

Wearing the label-like form under clothing cannot fully eliminate this problem. Junior-high-school and high-school boys typically are required in physical-education classes to undress completely to shower—thus revealing the label-like form and giving rise to the above-mentioned harassing or taunting behavior, perhaps even in aggravated form.

According to government studies, thirty to fifty percent of adolescents in substance-abuse treatment reported so-called recreational use of substances; accordingly such substances were added to a list of concerns. In 2004 it was reported that over five percent of high-school seniors engaged in illegal substance abuse, and as to the college level it has been estimated that as many as one in five college students have illegally practiced substance abuse.

Because legitimate use of many substances is both lawful and common, and the substances are easily available, even the direct discovery of certain substances in routine tests is of little value in identifying abusers. Throughout this document, where the term “substance” appears, to the extent appropriate to the context that term shall be understood to encompass any legally obtained substance susceptible to mechanical alteration for the purpose of snorting or injection.

Thus the threat posed by this type of substance abuse is extremely insidious. Yet it is a known killer.

### SUMMARY OF THE DISCLOSURE

The present invention significantly addresses this problem and provides much needed improvement. In preferred embodiments of a first of its aspects or facets the invention is a method for improving the safety of a substance at risk for abuse.

The method includes the step of preparing the substance in a form, other than a label-like form, that tends to deter conversion to powder. It also includes the step of providing the substance for commercial distribution in that form.

The foregoing may represent a description or definition of the first aspect or facet of the invention in its broadest or most general form. Even as couched in these broad terms, however, it can be seen that this facet of the invention importantly advances the art.

In particular, by preparing the substance in a form that tends to deter mechanical conversion to powder, the invention bears significant potential for reducing widespread abuse of common substances available on the market today, as mentioned above. A marked reduction may be expected.

For the reason explained in the preceding section of this document, a label-like form of the substance (such as is already on the market) cannot effectively prevent the abuse problems mentioned above. Therefore the foregoing definition or description expressly excludes such a label-like form of the substances mentioned above.

Although the first major aspect of the invention thus significantly advances the art, nevertheless to optimize enjoyment of its benefits preferably the invention is practiced in conjunction with certain additional features or characteristics. In particular, preferably the method further includes the step of, before the marketing step, enclosing the substance, in said form, in a small container that a person can harmlessly swallow. Another preference is that this method be applied specifically to a substance that is commonly abused, and is available reasonably readily.

Yet another preference is that the mechanical preparing step be performed by dissolving or dispersing the substance into or onto a small object that a person can harmlessly swallow. If this basic preference is observed, then it is further preferable that the object be:

water insoluble—whereby the mechanical preparing for the substance inhibits dissolving the mixture in water for injection; or

a solid—in which case, alternative preferences are that the solid be in the form of paper or other thin medium broadly extended in two dimensions; or in the form of a sponge or other medium having a generally coarse internal mechanical structure.

Other preferred forms are mentioned below.

In preferred embodiments of its second major independent facet or aspect, the invention is a product or composition of matter—in a particular, a mechanical preparation of a substance that is at risk for abuse, for improving the safety of that substance. The mechanical preparation includes the substance in a form (other than a label-like form) that tends to deter conversion to powder. It also includes an object containing the substance that form.

The foregoing may represent a description or definition of the second aspect or facet of the invention in its broadest or most general form. Even as couched in these broad terms, however, it can be seen that this facet of the invention importantly advances the art.

In particular, the benefits of this product aspect of the invention are closely related to those of the method aspect discussed above. These benefits particularly include reducing the availability of these substances in conditions that are susceptible to abuse—and thus reducing injuries and deaths caused by such abuse.

Although the second major aspect of the invention thus significantly advances the art, nevertheless to optimize enjoyment of its benefits preferably the invention is practiced in conjunction with certain additional features or characteristics. In particular, preferably the mechanical preparation of the substance can be placed in or on a small object that a person can harmlessly swallow. Other preferences described above for the first aspect of the invention are also applicable here.

In preferred embodiments of its third major independent facet or aspect, the invention is a commercial distribution method for improving the safety of a substance at risk for abuse. The method includes the steps of obtaining the substance in a form, other than a label-like form, that tends to deter conversion to powder.

The method also includes the step of selling the substance in such a form. The foregoing may represent a description or definition of the third aspect or facet of the invention in its broadest or most general form.

Even as couched in these broad terms, however, it can be seen that this facet of the invention importantly advances the art. In particular, the sale of commonly abused substances in a pulverization-deterring form enlists the commercial process in the aid of reducing rates and severity of substance abuse.

Although the third major aspect of the invention thus significantly advances the art, nevertheless to optimize enjoyment of its benefits preferably the invention is practiced in conjunction with certain additional features or characteristics. In particular, preferably the selling step includes wholesale selling; it also preferably includes retail selling.

Another preference is that the form include a mixture or suspension of the substance in or on a small object that a person can harmlessly swallow. If this preference is observed, then further preferences include obtaining the small object in the form of a paper, or other thin material broadly extended in two dimensions; or a sponge or other material having generally coarse internal mechanical structure.

The foregoing features and advantages of the invention will be more fully appreciated from the following Detailed Description, considered together with the accompanying drawings—of which:

### BRIEF DESCRIPTION OF THE DRAWINGS

FIG. 1 is a perspective or isometric view showing an inverted small bottle with a piece of substance placed thereon, as commonly done in the mechanical alteration of the subject substances for abuse purposes;

FIG. 2 is a like view showing a further stage in the abuse process;

FIG. 3 is another like view showing a final step; and

FIG. 4 is a flow chart representing very schematically the steps in practice of my invention.

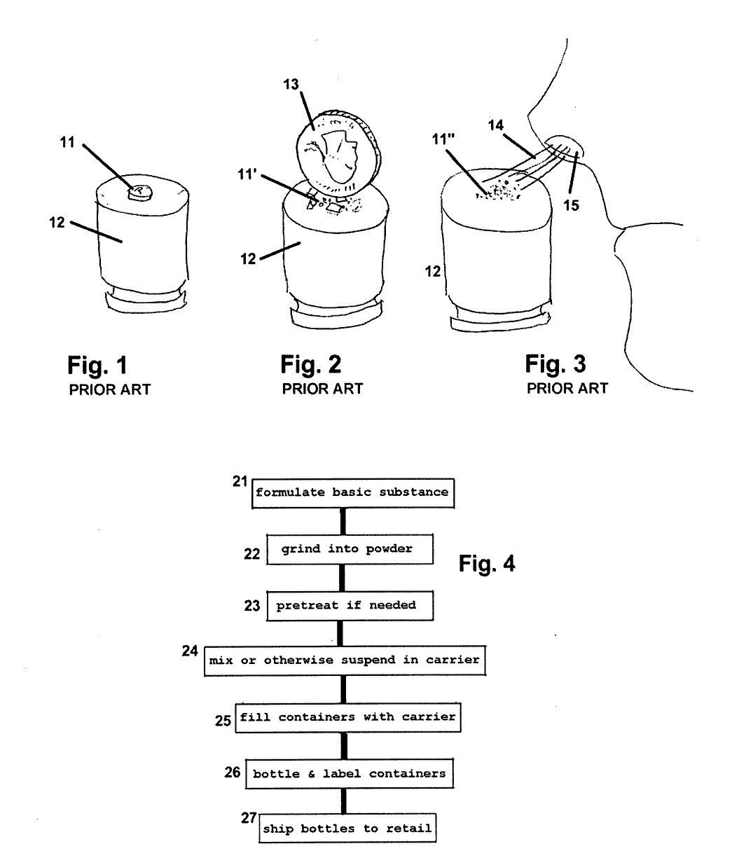

### DETAILED DESCRIPTION OF THE PREFERRED EMBODIMENTS

An objective of this invention, as will be clear from the foregoing discussion, is simply a tendency to deter physical or chemical separation of the substance from an object that can be harmlessly swallowed. To those skilled in this field, it will also be apparent that it is not absolute prevention that is required—to make practice of my invention extremely valuable to society—but rather only a meaningful tendency to deter.

Absolute deterrence or prevention could be desirable, but achieving that degree of abuse obstruction might be inordinately expensive or complex. Some people intent upon abusing or facilitating abuse of substances may have access to highly advanced physical or chemical separation facilities and abilities, such as are found in industrial or graduate-level laboratories. Extreme measures needed to preclude separation by such individuals may be impractical or produce new adverse consequences.

Rather, the degree of deterrence particularly sought through practice of the present invention is that which obstructs simple home-based methods such as are popular among casual abusers, e.g., as noted above, college students. The very great majority of these people have neither access to special means of separation nor an inclination to expend large sums of money to purchase such services.

Certain substances are widely made available in the U.S. and other countries for addressing various maladies, such as for example attention-deficit disorder. Unfortunately, these substances are frequently diverted for illicit use.

The commonest mode of abuse of such substances is snorting. The substance is ground into a fine dust and then rapidly inhaled through the nose, thereby introducing the substance powder widely onto the nasal membranes where it is rapidly absorbed into the bloodstream.

As noted above, this route of administration bypasses the natural processes of the stomach and liver. As a result, the effect of snorted substances is far more rapid and potent than the effect of the same amount of substance taken by mouth.

Snorting of many substances intended to be taken by mouth is dangerous for several reasons. First, the substances are usually mixed with other ingredients designed to improve the handling characteristics of the substance. These can include, according to the manufacturer, various dyes, conditioning ingredients etc. which may be injurious when snorted.

Second, because of the far more effective delivery of these substances into the body, there is a real risk of harmful results. These may particularly include powerful neurological effects and changes in heart rate and rhythm.

In particular the previously mentioned “chewable” product was not designed to prevent snorting, but rather merely for patients (particularly young children) who are unable to swallow pills intact.

The other new product discussed earlier is a label-like patch. The patch itself is snort proof; however, for at least two reasons it does not solve the abuse problems described above.

First, it does not entirely replace or supplant the common form of the substance, but instead leaves the latter on the market. Second, appearance of the label-like patch makes it obvious to anyone who sees the device that the child or other person uses the above named substance, subjecting the user to taunting, harassment, etc.

As noted above, wearing the label-like form under clothing cannot fully eliminate this problem. In competitive-athletics teams and most physical-education classes, junior-high-school and high-school boys typically are required to undress completely to shower—thus revealing the label-like form and giving rise to the above-mentioned harassing or taunting, perhaps even aggravated by the apparent effort at concealment.

It is possible and I believe likely that a child will remove the label-like article, to avoid such embarrassments—or to sell it to a peer who wants a sensation (“kick”) which the substance may produce.

The government reports that thirty to fifty percent of adolescents in substance treatment reported so-called “illegitimate” use of certain substances (Ritalin, for example). Official studies were conducted in 2004 and 2005 showing that over five percent of high-school seniors abuse dangerous and harmful substances, and as to the college level estimated that as many as one in five college students have used such substances illegally.

In accordance with preferred embodiments of my invention, legitimate substances meant to ingested by mouth are reformulated into a small object that can be safely swallowed by a person containing the active substance—and whatever other ingredients might be of value in this novel mixture—all prepared as or dissolved in an easy-to-swallow and harmless small object. The nature of the object is chosen to deter snorting and other modes of abuse mentioned above.

Thus the object may comprise a liquid, in which the substance is simply suspended or preferably dissolved; or may comprise a carrier that is a gel, or alternatively a solid. A particularly advantageous liquid is one—such as (merely by way of example) an oil—from which the substance is awkward or difficult to remove without special equipment or training, or preferably both. Liquids from which the substance can be mechanically recovered simply by e.g. drying, evaporation or settling, should not be used.

Although for some purposes possibly it may seem more advantageous to select a carrier of the type mentioned above rather than liquid in general, some particular liquids may be very effective. With respect to certain objects, the substance should be adhered to (as for example by gluing) or embedded within a solid—such as paper, sponge, or a gelatinous etc. confection or pastry—one which is not easy to grind.

The object should be chosen to avoid interfering with the desired legitimate use of the substance, when the object and the substance together are taken by mouth in the approved usual way. This is particularly noteworthy in the case of a solid object such as paper, sponge, etc., since the selected solid must be one that can be chewed, or in any event harmlessly swallowed and digested, notwithstanding its incompatibility with mechanical grinding.

If the substance is placed as small particles through-out or on the object, the combination can be called a two-phase system. If the substance appears to be completely dissolved in the object, the resulting clear or colored mixture can be termed a one-phase system.

The most difficult manufacturing problem with a mixture that includes such a carrier substance is ensuring that the substance is distributed evenly, to a reasonable degree, throughout the carrier and substantially without clumping. This may be accomplished by:
smoothly sifting the powdered substance into the rapidly stirred carrier,
making a smooth paste of the substance with a water-miscible agent such as an alcohol or propylene glycol, or
simply using a blender to completely homogenize the mixture of carrier and powder.

Generally these substances, which are commonly used ingredients, are freely soluble in water, and therefore should easily make such a carrier mixture suitable for such combination. The several object ingredients mentioned above include many that are already regulatorily approved; thus the approval requirements for at least initial practice of this invention should be minimal.

I believe that availability of suitable carriers is not a problem, because many gels suitable for use in practicing this invention are inert. Cellulose-based such carriers are routinely used as inert thickeners in various food products.

In any event, the object with the substance in it is placed or molded into an object (e.g. capsule) of, ideally, a commonly used type of appropriate material—or a material with closely related characteristics. Such objects, as is well known, in common use dissolve rapidly to release the substance into the body—and this should be the case in practice of the present invention as well.

Given suitable choice of an object that can be harmlessly swallowed, the resulting combination of the substance and object, enclosed in or molded as a combination, should have essentially the same physiological effects as the common form of the substance.

Snorting of the object contents will, however, be substantially impossible. Physical properties of a carrier such as described above—e.g. a sticky semifluid—prevent breaking the substance into a fine aerosol and thus block its wide mechanical introduction into the nose. Separating substance from object, though possible in a specially equipped lab, would be difficult otherwise.

To make the mixture resistant to injection abuse, the substance should be placed into such a carrier or other material that is not soluble in water as part of a two-phase system. Given this precaution, the object contents will not be easily converted into injection form.

Thus my invention is advantageously practiced by the steps comprising: mixing 21 (FIG. 4) the basic substance, grinding 22 the mixed substance into powder, applying any pretreatment 23 that may be needed, and mixing or otherwise suspending 24 the substance into a carrier of any of the types mentioned above. Thereafter, the invention may comprise the further steps of filling 25 objects with the substance-containing carrier, bottling and labeling the objects 26, and dispensing the objects for marketing 27.

The foregoing disclosure is intended to be merely exemplary and not to limit the scope of the invention—which is defined by the appended claims.

I claim:

1. A method for improving the safety of a substance at risk for abuse; said method comprising the steps of:
formulating the substance into a form, other than a label-like form, that tends to deter conversion to powder; and
providing the substance for commercial distribution in said form.

2. The method of claim 1, further comprising the step of:
before the marketing step, enclosing the substance, in said form, in a form that can easily and harmlessly be swallowed by a person.

3. The method of claim 1, wherein:
the mixing step is performed by dissolving or dispersing the substance into or onto a form that can harmlessly be swallowed by a person.

4. The method of claim 1, wherein:
the object is water-insoluble;
whereby the mixture inhibits dissolving the substance in water for injection.

5. The method of claim 1, wherein:
the object is a solid.

6. The method of claim 5, wherein:
the solid is in the form of paper or other thin medium broadly extended in two dimensions.

7. The method of claim 5, wherein:
the solid is in the form of a sponge or other medium having a generally coarse cellular structure.

8. A substance mixture for improving the safety of a substance at risk for abuse; said mixture comprising:
the substance, in a form, other than a label-like form, that tends to deter conversion to powder; and
an object containing the substance in said form.

9. The formulation of claim 8, wherein:
said form comprises a mixture of the substance in or on an object that can be harmlessly swallowed by a person.

10. A commercial distribution method for improving the safety of a substance at risk for abuse; said method comprising the steps of:
obtaining the substance in a form, other than a label-like form, that tends to deter conversion to powder; and
selling the substance in said form.

11. The method of claim 10, wherein:
the selling step comprises wholesale selling.

12. The method of claim 10, wherein:
the selling step comprises retail selling.

13. The method of claim 10, wherein:
said form comprises a mixture of the substance in or on an object that can harmlessly be swallowed by a person.

14. The method of claim 13, wherein:
the object is in the form of a paper, or other thin material broadly extended in two dimensions; or a sponge or other material having generally coarse cellular structure.
